(19) United States
(12) Patent Application Publication
(10) Pub. No.: US 2022/0031739 A1
(43) Pub. Date: Feb. 3, 2022

(54) ANTI-VIRAL COMPOSITIONS AND METHODS OF USE THEREOF

(71) Applicant: Eye Therapies LLC, Dana Point, CA (US)

(72) Inventor: Gerald Horn, Deerfield, IL (US)

(21) Appl. No.: 17/389,556

(22) Filed: Jul. 30, 2021

Related U.S. Application Data
(60) Provisional application No. 63/059,231, filed on Jul. 31, 2020.

(51) Int. Cl. A61K 33/30; A61K 31/4168; A61K 31/137; A61K 31/498; A61K 31/155; A61K 31/198; A61K 31/4164; A61K 31/4178; A61K 47/32; A61K 47/44; A61K 47/38; A61K 47/26; A61K 47/18

(52) U.S. Cl. CPC A61K 33/30; A61K 31/4168; A61K 31/137; A61K 31/498; A61K 31/155; A61K 31/198; A61K 47/183; A61K 31/4178; A61K 47/32; A61K 47/44; A61K 47/38; A61K 47/26; A61K 31/4164

(57) ABSTRACT

The present invention is directed to anti-viral compositions comprising zinc, optionally, an alpha-adrenergic receptor agonist, one or more nonionic surfactants and one or more viscosity enhancers. The present invention is further directed to methods of treating viral infections comprising administering compositions of the present invention to a subject in need thereof.

### FIELD OF THE INVENTION

The present invention is directed to anti-viral compositions comprising zinc, optionally, an alpha-adrenergic receptor agonist, one or more nonionic surfactants and one or more viscosity enhancers. The present invention is further directed to methods of treating viral infections comprising administering compositions of the present invention to a subject in need thereof.

### BACKGROUND OF THE INVENTION

Viral infections of the eye range from the often-benign viral conjunctivitis to the much more severe herpes keratitis. While usually benign, viral conjunctivitis is uncomfortable and visually displeasing. Viral conjunctivitis is also the most common eye disease with an estimated 6 million cases of viral conjunctivitis occurring in the United States each year.

Herpes keratitis occurs in about 1.5 million cases worldwide each year. Of those infected nearly 40,000 result in severe visual impairment or blindness. In fact, herpes keratitis is the most common cause of blindness due to corneal disease in developed nations.

There are only a few treatments for viral infections of the eye currently on the market. Viroptic®, which contains 1% trifluridine, is approved to treat epithelial keratitis caused by herpes simplex virus (“HSV”). (Viroptic is a registered trademark of and available from Monarch Pharmaceuticals). Trifluridine is a nucleoside analog. Trifluridine can treat superficial HSV infections within 1 week from the start of treatment. However, because trifluridine is a nucleoside analog which is incorporated into DNA, trifluridine causes toxicity of both virus and corneal cells. The likelihood of toxicity is especially high when used for long periods for deeper infections.

Another treatment for viral infections of the eye is acyclovir, which is approved in Europe for topical administration to treat ocular herpetic keratitis. However, acyclovir formulations are poorly tolerated ointments that interfere with vision. Further, topical acyclovir is not approved outside Europe for ophthalmic use.

The latest treatment for viral eye infections is Zirgan®, which contains 0.15% ganciclovir, and is approved for the treatment of acute herpetic keratitis (Zirgan is a registered trademark of Laboratoires Thea Société Par Actions Simplifiée and is available from Bausch & Lomb).

COVID-19 has spread across the world. COVID-19 is caused by the coronavirus strain SARS-CoV-2 and may cause severe respiratory distress leading to death. Prior to the spread of COVID-19 other coronavirus strains caused respiratory diseases such as SARS and MERS. To date, there is no effective treatment capable of inhibiting the growth and spread of coronavirus in humans.

Thus, there is a need in the art for anti-viral composition that can be used to treat ocular and respiratory viral infections. Preferably, this anti-viral composition would cause relatively low or no side effects and be well tolerated.

### SUMMARY OF THE INVENTION

The present invention is directed to anti-viral compositions comprising zinc, one or more nonionic surfactants and one or more viscosity enhancers.

The present invention is further directed to methods of treating viral infections comprising administering compositions of the present invention to a subject in need thereof.

### DETAILED DESCRIPTION OF THE INVENTION

In one embodiment, the present invention is directed to anti-viral compositions comprising zinc, an alpha-adrenergic receptor agonist, one or more nonionic surfactants and one or more viscosity enhancers.

In one embodiment, the present invention is directed to anti-viral compositions comprising an alpha-adrenergic receptor agonist, zinc, one or more nonionic surfactants and one or more viscosity enhancers.

Zinc may be present in compositions of the present invention as a salt or in a chromophore.

Zinc salts suitable for use in the present invention include, but are not limited to, hydrochloride, hydrochloride dihydrate, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, glycinate, saccharate, formate, benzoate, glutamate, methanesulfonate, ethanesulfonate, benzensulfonate, p-toluenesulfonate, oxide and pamoate (i.e., 1,1′-methylene-bis-(2-hydroxy-3-naphthoate)) salts. In a preferred embodiment, the zinc salt is zinc acetate, zinc citrate, zinc glycinate, zinc sulfate or zinc oxide. Zinc salts may be present in compositions of the present invention at a concentration from about 0.01% to about 5% w/v, preferably from about 0.1% to about 3.0% w/v and more preferably from about 0.2% to about 2.5% w/v and most preferably at about 0.25%, 0.5%, 0.75%, 1.0% or 2.3% w/v.

Alpha-adrenergic receptor agonists suitable for use in the present invention include, but are not limited to, apraclonidine, clonidine, tetrahydrozoline, naphazoline, oxymetazoline, xylometazoline, methoxamine, epinephrine, norepinephrine, phenylephrine, brimonidine, guanfacine, fadolmidine, dexmedetomidine, (+)-(S)-4-[1-(2,3-dimethyl-phenyl)-ethyl]-1,3-dihydro-imidazole-2-thione, 1-[(imidazolidin-2-yl)imino]indazole and methyl dopa. In a preferred embodiment, the alpha-adrenergic receptor agonist is oxymetazoline or brimonidine. Alpha-adrenergic receptor agonists may be present in compositions of the present invention at a concentration from about 0.0001% to about 1% w/v, preferably from about 0.0005% to about 0.5% w/v, more preferably from about 0.001% to about 0.5% w/v, even more preferably from about 0.001% to about 0.1% w/v even more preferably from about 0.01% to about 0.1% w/v, yet even more preferably from about 0.02% to about 0.06% w/v and most preferably at about 0.025% or 0.02% w/v.

Nonionic surfactants suitable for use in the present invention include, but are not limited to a polysorbate, a poloxamer, a polyoxyl, an alkyl aryl poly ether, a cyclodextrin, a tocopheryl, polyethylene glycol succinate a glucosyl dialkyl ethers and a crown ether, ester-linked surfactants. Nonionic surfactants may be present in compositions of the present invention at a concentration from about 1% to about 10% w/v and preferably from about 1.5% to about 7.0% w/v.

Polysorbates suitable for use in the present invention include, but are not limited to, polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) and polysorbate 80 (polyoxyethylene (20) sorbitan monooleate.

Cyclodextrins suitable for use in the present invention include, but are not limited to, ionically charged (e.g. anionic) beta-cyclodextrins with or without a butyrated salt (Captisol) 2-hydroxypropyl beta cyclodextrin (“HPβCD”), alpha cyclodextrins and gamma cyclodextrins.

Poloxamers include but are not limited to poloxamer 103, poloxamer 123, and poloxamer 124, poloxamer 407, poloxamer 188, poloxamer 338 and any poloxamer analogue or derivative.

In a preferred embodiment, the poloxamer is poloxamer 407. Poloxamers may be present in compositions of the present invention at a concentration from about 1% to about 5% w/v, preferably from about 1% to about 4% w/v, more preferably from about 2% to about 3% w/v and most preferably at about 2.5% w/v.

Polyoxyls include but are not limited to polyoxyl castor oils such as polyoxyl 35 castor oil, polyoxyl 40 castor oil, polyoxyl 45 castor oil, Brij® 35, 78, 98, 700 (polyoxyethylene glycol alkyl ethers) and Spans (sorbitan esters) and Span® 20-80 (sorbitan monolaurate, sorbitan monopalmitate, sorbitan monostearate, and sorbitan monooleate). In a preferred embodiment, the polyoxyl is a polyoxyl castor oil. Polyoxyls may be present in compositions of the present invention at a concentration from about 0.01% to about 1% w/v, preferably from about 0.1% to about 0.5% w/v, more preferably from about 0.2% to about 0.3% w/v and most preferably at about 0.25% w/v.

Viscosity enhancers suitable for use in the present invention include, but are not limited to, cellulose derivatives such as hydroxypropylmethyl cellulose (“HPMC”), hydroxypropyl cellulose and carboxymethyl cellulose (“CMC”), carbomers, gums and hyaluronates. In a preferred embodiment the viscosity enhancer is a cellulose derivative. Viscosity enhancers may be present in compositions of the present invention at a concentration from about 0.1% to about 5% w/v, preferably from about 0.1% to about 2% w/v or from about 1% to about 3% w/v, and more preferably from about 1% to about 2% w/v and most preferably at about 1.25% or 1.4% w/v.

Viscosity enhancer may further be present in compositions of the invention at a concentration that provides a zero shear viscosity of from about 50 to about 1,000 centipoise (“cps”), preferably from about 100 to about 500 cps and more preferably from about 200 to about 400 cps.

In a preferred embodiment, the compositions of the present invention further comprise one or more excipients selected from the group consisting of one or more oils, one or more polyols, one or more antioxidants, one or more preservatives, one or more buffers, L-cysteine, N-acetyl cysteine, sodium chloride, and a sorbate.

Oils suitable for use in the present invention include, but are not limited to, castor oil, linoleic oil, sesame oil and the like. Oils may be present in compositions of the present invention at a concentration from about 0.0001% to about 99.999% w/v, preferably from about 0.1% to about 99.9% w/v and more preferably from about 1% to about 99% w/v.

Polyols suitable for use in the present invention include, but are not limited to, mannitol, glycerol, erythritol, lactitol, xylitol, sorbitol, isosorbide, and maltitol. In a more preferred embodiment, the polyol is mannitol. In another more preferred embodiment, the polyol is at a concentration from about 0.1% to about 5.5% w/v, preferably from about 0.25% to about 5% w/v, more preferably from about 1% to about 5% w/v, even more preferably from about 1.5% to about 2.5% w/v, and most preferably at about 2% w/v.

Antioxidants suitable for use in the present invention include, but are not limited to, ascorbic acid, astaxanthin, citrate, EDTA, sodium metabisulfite, sodium thiosulfate, acetylcysteine, butylated hydroxyanisole and butylated hydroxytoluene and a combination thereof. In a preferred embodiment, the antioxidant is sodium metabisulfite. Antioxidants may be present in compositions of the present invention at a concentration from about 0.001% to about 1% w/v, preferably from about 0.01% to about 0.1% w/v, more preferably from about 0.025% to about 0.075% w/v and most preferably at about 0.05% w/v.

Preservatives suitable for use in the present invention include, but are not limited to, benzalkonium chloride (“BAK”), sorbate, EDTA, methyl paraben or peroxide based preservatives. In a preferred embodiment, the preservative is EDTA. Preservatives may be present in compositions of the present invention at a concentration from about 0.01% to about 1% w/v, preferably from about 0.01% to about 0.5% w/v, more preferably from about 0.05% to about 0.2% w/v and most preferably at about 0.1% w/v.

Buffers useful in the present invention include, but are not limited to, citrate buffer, acetate buffer, borate buffer and phosphate buffer. Buffers may be present in compositions of the present invention at a concentration from about 1.0 millimolar (“mM”) to about 10.0 mM, preferably from about 4.0 mM to about 10.0 mM and more preferably at about 4.5, 5.0, 6.0, 7.5 or 10.0 mM.

L-cysteine may be present in compositions of the present invention at a concentration from about 0.001% to about 1% w/v, preferably from about 0.01% to about 0.1% w/v, more preferably from about 0.025% to about 0.075% w/v and most preferably at about 0.05% w/v.

N-acetyl cysteine may be present in compositions of the present invention at a concentration from about 0.001% to about 1% w/v, preferably from about 0.01% to about 0.1% w/v, more preferably from about 0.025% to about 0.075% w/v and most preferably at about 0.05% w/v.

Sodium chloride may be present in compositions of the present invention at a concentration from about 0.1% to about 2% w/v, preferably from about 0.2% to about 1.0% w/v, more preferably from about 0.6% to about 0.8% w/v and most preferably at about 0.7% w/v.

Sorbate may be present in compositions of the present invention at a concentration from about 0.01% to about 0.5% w/v, preferably from about 0.05% to about 0.25% w/v and more preferably at about 0.12% w/v. In a preferred embodiment the sorbate is potassium sorbate.

Compositions of the present invention may have a pH from about 3.0 to about 9.0, more preferably from about 4.5 to about 8.0 or from about.5.5 to about 8.0, even more preferably from about 7.5 to about 8.0 and even more preferably from about 7.7 to about 7.8, and most preferably at about 7.75.

The compositions of the present invention may in the form of a liquid, gel, ointment or cream. Compositions of the present invention may be applied to the eye of a subject in need thereof by any available means including, but not limited to, a drop or a spray. The subject may be wearing a contact lens during instillation of the compositions of the present invention. Compositions of the present invention may also be placed on a contact lens that is then inserted in the eye of a subject in need thereof.

The compositions of the present invention may also be administered via the respiratory system of a subject in need thereof including nose, mouth, throat and lungs. Administration to the lungs may be occur as an aerosolized spray such as via a nebulizer, an inhaler or the like. Administration to the throat may be occur in the form of a syrup, a spray, a gel or the like.

In another embodiment, the present invention is further directed to methods of treating viral infections comprising administering compositions of the present invention to a subject in need thereof.

Viral infections that may be treated by administration of the compositions of the present invention include, but are not limited to, retinitis, conjunctivitis, keratitis, keratoconjunctivitis, uveitis, ocular herpes including herpes zoster ophthalmicus, pharyngitis, rhinitis, sinusitis, bronchitis, pulmonitis, pneumonia, influenza, COVID-19, SARS and MERS.

Viral infections that may be treated by administration of the compositions of the present invention include infections caused by a variety of viruses including, but not limited to, herpes viruses including herpes simplex virus, cytomegalovirus, vesicular stomatitis virus, varicella zoster virus, human herpesvirus 6, and Epstein-Barr virus and adenovirus, human immunodeficiency virus, pox virus, rubeola virus influenza virus and coronavirus.

As used herein, all numerical values relating to amounts, weights, and the like, that are defined as “about” each particular value is plus or minus 10%. For example, the phrase “about 5% w/v” is to be understood as “4.5% to 5.5% w/v.” Therefore, amounts within 10% of the claimed value are encompassed by the scope of the claims.

As used herein “% w/v” refers to the percent weight of the total composition.

As used herein the terms “subject” and “patient” are used interchangeably and refer, but are not limited to, a person or other animals.

As used herein, the term “prevent” or “preventing” refers to precluding, averting, obviating, forestalling, stopping, or hindering something from happening, especially by advance action.

As used herein, the term “treat” or “treating” refers to reversing, alleviating or slowing the progress of the disease, disorder, or condition to which such terms apply, or one or more symptoms of such disease, disorder, or condition.

As used herein, the term “administration” or “administering” refers to application to the ocular or respiratory system of the subject. Administration may occur prophylactically or in response to symptoms.

The articles “a,” “an” and “the” are intended to include the plural as well as the singular, unless the context clearly indicates otherwise.

In a preferred embodiment, the present invention is directed to anti-viral compositions comprising oxymetazoline, poloxamer 407, polyoxyl castor oil, a zinc salt, carboxymethyl cellulose, EDTA, L-cysteine, N-acetyl cysteine, sodium metabisulfite, a buffer and potassium sorbate.

In another preferred embodiment, the present invention is directed to anti-viral compositions comprising brimonidine, poloxamer 407, polyoxyl castor oil, a zinc salt, carboxymethyl cellulose, EDTA, L-cysteine, N-acetyl cysteine, sodium metabisulfite, a buffer and potassium sorbate.

In a more preferred embodiment, the present invention is directed to anti-viral compositions comprising about 0.02% w/v brimonidine or oxymetazoline, about 2.5% w/v poloxamer 407, about 0.25% w/v polyoxyl castor oil, about 0.25% w/v zinc citrate or zinc sulfate, about 1.40% w/v CMC or about 1.25% HPMC, about 0.10% w/v EDTA, about 0.05% w/v L-cysteine, about 0.05% w/v N-acetyl cysteine, about 0.05% w/v sodium metabisulfite, optionally, about 4.5, 5.0, 6.0, 7.5 and 10.0 mM borate buffer and optionally about 0.10% w/v potassium sorbate.

In another more preferred embodiment, the present invention is directed to anti-viral compositions comprising about 0.02% w/v oxymetazoline, about 2.5% w/v of a polysorbate, about 2.3% w/v zinc acetate, about 1.25% w/v hydroxypropylmethyl cellulose, about 0.7% w/v sodium chloride, about 2% w/v mannitol and optionally about 2.5 mM citate buffer, wherein the compositions have a pH of about 5.7.

### EXAMPLES

### Example 1

### Prophetic Treatment of Healthy Individuals with Composition of the Present Invention

### Method

20 Healthy mammals with no one evidence of any disease state were given 50 microliters of either a composition of the present invention as an eye drop, nasal spray, throat spray, and nebulized mist twice daily. A control group received the same administration of a saline solution.

2 days following the last treatment the eyes of the mammal were examined for symptoms of eye infection and tested for viral load.

### Results

The 20 mammals treated with the composition of the present invention had no clinical or histopathologic abnormality. The 20 mammals treated with saline had identical outcomes. Thus, the compositions of the present invention present no significant adverse events or toxicity.

### Example 2

### Prophetic Treatment of Viral Eye Infection with Composition of the Present Invention

### Method

The eyes of mammals (preferably a rabbit or a primate) were infected bilaterally with a high topical surface exposure of virus load of SARS-CoV-2 2 days following initiation of prophylactic BID ophthalmic treatment of ET-ZA23 ophthalmic drops continued for 14 days. 50 microliters of a saline solution was administered into both eyes of a control group mammal on the same schedule of exposure to virus topically 2 days following initiation of saline drops.

2 days following the last treatment the eyes of the mammal were examined for symptoms of eye infection and tested for viral load.

### Results

The eyes of the mammals treated with a composition of the present invention had little to no signs of an infection. However, 90% of the eyes treated with saline solution presented with active virus identified present topically on the ocular surface and ophthalmic signs of peripheral vascular injection.

Only 5% of the 20 mammals treated with the composition of the present invention demonstrated moderate infection. One mammal treated with the composition of the present invention demonstrated severe infection. However, in the control group 25% developed moderate infection and 60% developed severe infection with several deaths.

The mammals treated with a composition of the present invention had extremely reduced viral load as compared to the mammals treated with ophthalmic saline solution prior to delivery of the topical ocular high viral exposure. Thus, compositions of the present invention are a highly effective treatment against viral infections of the eye.

### Example 3

### Prophetic Treatment of Viral Throat and Nasal Infection with Composition of the Present Invention

### Method

The nasal turbinates and throat of mammals (preferably a rabbit or a primate) were infected with a virus 2 days following initiation of BID prophylactic nasal and throat spray treatment of approximately 50 microliters per spray of a composition of the present invention. This treatment continued 2 times daily for 2 weeks. 50 microliters of a saline solution was administered on an identical regimen to controls. A nasal spray tapered tip allowed focal delivery to the targets of upper nasal turbinates as well as throat.

2 days following the last treatment the mammal were examined for symptoms of local and systemic COVID-19 infection and tested for viral load.

### Results

25% of the mammals treated with a composition of the present invention exhibited mild to moderate signs of an infection. However, 70% of the mammals treated with saline solution exhibited moderate to severe infection and 5% expired. Further, the mammals treated with a composition of the present invention had extremely reduced viral load as compared to the mammals treated with saline solution and no pulmonary of infection. However, of those treated with saline, 90% exhibited significant viral load and 25% had multisystem organ pathology particularly to brain, lung, heart, and kidneys. Thus, compositions of the present invention are a highly effective prophylaxis treatment against viral infections of the throat and nasal passages.

### Example 4

### Prophetic Treatment of Repeat Inoculation of Controls in Examples 2 and 3 Exhibiting Systemic Disease with Nebulized Composition Sprays of the Present Invention

### Method

The experiments of Examples 2 and 3 were repeated in control groups that were administered 50 microliters of a saline solution to the ocular surface in Example 2 and throat and upper nasal turbinates in Example 3 as well as aerosolized exposure of these mammals to virus. No compositions of the present invention were administered prior to the control mammals exhibiting onset of systemic disease. Once control mammals exhibited systemic disease, they were randomly assigned to receive either:

Twice a day: topical drops, nasal and throat spray, and nebulized pulmonary delivery including 2-micron droplets of a composition of the present invention for 4 weeks; or
Twice a day: topical drops, nasal and throat spray, and nebulized pulmonary delivery including 2-micron droplets of saline for 4 weeks.

2 days following the last treatment the mammal were examined for symptoms of local and systemic COVID-19 infection and tested for viral load.

### Results

Of the 20 mammals treated with a composition of the present invention, 5% died and 25% exhibited mild to moderate signs of an infection. Of the 20 animals treated with a composition of the present invention, 50% exhibited no virus load, 45% exhibited mild virus load and only 5% exhibited significant virus load. Only 5% of the 20 mammals treated with a composition of the present invention exhibited brain, heart or lung damage. Of the 20 animals treated with saline, 25% died. Of the 20 animals treated with saline, 100% exhibited significant viral loads of which 75% were severe. 80% of the saline treated mammals had organ damage to one or more of brain, heart, lung or kidneys. Thus, compositions of the present invention are a highly effective treatment against viral infections initiated via the eye, throat, nasal passages, and pulmonary inhalation when administered post onset of clinical signs and symptoms via a combination of topical ophthalmic, nasal and throat spray, and nebulized administration twice daily for 30 days.

What is claimed is:

1. An anti-viral composition comprising zinc, one or more nonionic surfactants and one or more viscosity enhancers.

2. The composition of claim 1 further comprising an alpha-adrenergic receptor agonist,

3. The composition of claim 2, wherein the alpha-adrenergic receptor agonist is selected from the group consisting of apraclonidine, clonidine, tetrahydrozoline, naphazoline, oxymetazoline, xylometazoline, methoxamine, epinephrine, norepinephrine, phenylephrine, brimonidine, guanfacine, fadolmidine, dexmedetomidine, (+)-(S)-4-[1-(2,3-dimethyl-phenyl)-ethyl]-1,3-dihydro-imidazole-2-thione, 1-[(imidazolidin-2-yl)imino]indazole and methyl dopa.

4. The composition of claim 3, wherein the alpha-adrenergic receptor agonist is oxymetazoline or brimonidine.

5. The composition of claim 1, wherein the zinc is present as a zinc salt selected from the group consisting of hydrochloride, hydrochloride dihydrate, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, glycinate, saccharate, formate, benzoate, glutamate, methanesulfonate, ethanesulfonate, benzensulfonate, p-toluenesulfonate, oxide and pamoate.

6. The composition of claim 5, wherein the zinc salt is selected from the group consisting of acetate, citrate, glycinate, oxide and sulfate.

7. The composition of claim 1, wherein the one or more nonionic surfactants are selected from the group consisting of a polysorbate, a poloxamer, a polyoxyl, an alkyl aryl poly ether, a cyclodextrin, a tocopheryl, polyethylene glycol succinate a glucosyl dialkyl ethers and a crown ether, ester-linked surfactant.

8. The composition of claim 7, wherein the one or more nonionic surfactants are a poloxamer and a polyoxyl castor oil.

9. The composition of claim 1, wherein the one or more viscosity enhancers are selected from the group consisting of cellulose derivatives, carbomers, gums and hyaluronates.

10. The composition of claim 9, wherein the one or more viscosity enhancers is carboxymethyl cellulose or hydroxypropylmethyl cellulose.

11. The composition of claim 1 further comprising one or more excipients selected from the group consisting of one or more oils, one or more polyols, one or more antioxidants, one or more preservatives, one or more buffers, L-cysteine, N-acetyl cysteine, sodium chloride, and a sorbate.

12. The composition of claim 11, wherein the one or more oils is selected from the group consisting of castor oil, linoleic oil and sesame oil.

13. The composition of claim 11, wherein the one or more polyols is selected from the group consisting of mannitol, glycerol, erythritol, lactitol, xylitol, sorbitol, isosorbide, and maltitol.

14. The composition of claim 11, wherein the one or more antioxidants are selected from ascorbic acid, astaxanthin citrate, EDTA, sodium metabisulfite, sodium thiosulfate, acetylcysteine, butylated hydroxyanisole and butylated hydroxytoluene.

15. The composition of claim 11, wherein the one or more preservatives are selected from the group consisting of benzalkonium chloride, sorbate, EDTA, methyl paraben or a peroxide-based preservative.

16. The composition of claim 11, wherein the one or more buffers are selected from the group consisting of citrate buffer, acetate buffer, borate buffer and phosphate buffer.

17. An anti-viral composition comprising:
from about 0.01% to about 5.0% w/v zinc;
from about 0.001% to about 0.1% w/v of an alpha-adrenergic receptor agonist;
from about 1% to about 5% w/v of one or more nonionic surfactants;
from about 0.1% to about 5% w/v of one or more viscosity enhancers;
from about 0.01% to about 0.1% w/v of one or more antioxidants;
from about 0.01% to about 0.5% w/v of one or more preservatives;
from about 0.01% to about 0.1% w/v of L-cysteine;
from about 0.01% to about 0.1% w/v of N-acetyl cysteine;
from about 4.0 to about 10.0 millimolar of a buffer; and
optionally, from about 0.05% to about 0.25% w/v of a sorbate,
wherein w/v denotes weight by total volume of the composition.

18. The composition of claim 17 comprising:
about 0.02% w/v brimonidine or oxymetazoline;
about 0.25% w/v zinc citrate or zinc sulfate;
about 2.5% w/v poloxamer 407;
about 0.25% w/v polyoxyl castor oil;
about 1.40% w/v carboxymethyl cellulose or about 1.25% w/v hydroxypropylmethyl cellulose;
about 0.10% w/v EDTA;
about 0.05% w/v L-cysteine;
about 0.05% w/v N-acetyl cysteine;
about 0.05% w/v sodium metabisulfite; and
optionally, about 0.12% w/v sorbate,
wherein w/v denotes weight by total volume of the composition.

19. An anti-viral composition comprising:
from about 0.001% to about 0.5% w/v oxymetazoline,
from about 0.01% to about 5.0% w/v of a zinc salt;
from about 1.25% to about 4.0% w/v of a polysorbate;
from about 1% to about 5% w/v of one or more polyols;
from about 0.1% to about 5% w/v of one or more viscosity enhancers;
from about 0.1% to about 2% w/v sodium chloride;
from about 1.0 to about 5.0 mM of one or more buffers, wherein the composition has a pH from about 4.5 to about 8.0.

20. The composition of claim 19 comprising:
about 0.2% w/v oxymetazoline;
about 2.3% w/v zinc acetate;
about 2.5% w/v of a polysorbate;
about 1.25% w/v hydroxypropylmethyl cellulose;
about 2% w/v mannitol;
about 0.7% w/v sodium chloride;
about 2.5 mM citrate buffer,
wherein the composition has a pH of about 5.7.

21. A method of treating a viral infection comprising administering a composition of claim 1.

22. The method of claim 21, wherein the viral infection is selected from the group consisting of retinitis, conjunctivitis, keratitis, keratoconjunctivitis, uveitis, pharyngitis, rhinitis, sinusitis, bronchitis, pulmonitis, pneumonia, ocular herpes, influenza, COVID-19, SARS and MERS.

23. The method of claim 21, wherein the viral infection is caused by a virus selected from the group consisting of herpes simplex virus, cytomegalovirus, vesicular stomatitis virus, varicella zoster virus, human herpesvirus 6, and Epstein-Barr virus and adenovirus, human immunodeficiency virus, pox virus, rubeola virus influenza virus and coronavirus.
